(19) United States
(12) Patent Application Publication
(10) Pub. No.: US 2025/0001219 A1
(43) Pub. Date: Jan. 2, 2025

(54) TRANSCRANIAL FOCUSED ULTRASOUND ACOUSTIC PRESSURE FIELD PREDICTION DEVICE, TRANSCRANIAL FOCUSED ULTRASOUND ACOUSTIC PRESSURE FIELD PREDICTION PROGRAM, AND METHOD FOR IMPLEMENTING ACOUSTIC PRESSURE FIELD GENERATION ARTIFICIAL INTELLIGENCE

(71) Applicant: KOREA UNIVERSITY RESEARCH AND BUSINESS FOUNDATION, Seoul (KR)

(72) Inventors: Gun Woo NOH, Seoul (KR); Kyung Ho YOON, Seoul (KR); Min Wook CHOI, Seoul (KR); Min Yeong JANG, Seoul (KR); In Su JEONG, Namyangju-si (KR)

(73) Assignee: KOREA UNIVERSITY RESEARCH AND BUSINESS FOUNDATION, Seoul (KR)

(21) Appl. No.: 18/578,557

(22) PCT Filed: Oct. 31, 2022
(86) PCT No.: PCT/KR2022/016845

§ 371 Date: Jan. 11, 2024

(30) Foreign Application Priority Data
Sep. 28, 2022 (KR) 10-2022-0123592

(51) Int. Cl. A61N 7/02; A61N 7/00

(52) U.S. Cl. CPC A61N 7/02; A61N 2007/0026

(57) ABSTRACT

The present invention relates to a transcranial focused ultrasound acoustic pressure field prediction device, a transcranial focused ultrasound acoustic pressure field prediction program, and a method for implementing acoustic pressure field generation artificial intelligence. The transcranial focused ultrasound acoustic pressure field prediction device includes: an input/output unit for allowing a user to input data, and outputting the data in a form that is recognizable by the user, a memory for storing a transcranial focused ultrasound acoustic pressure field prediction program; and a control unit for executing the transcranial focused ultrasound acoustic pressure field prediction program to derive result data according to the data input through the input/output unit, wherein the control unit outputs ultrasound acoustic pressure field data formed in a transcranial region by ultrasound applied by an ultrasonic transducer when shape data of a skull and position data of the ultrasonic transducer are input.

### TECHNICAL FIELD

The present invention relates to a transcranial focused ultrasound acoustic pressure field prediction device, a transcranial focused ultrasound acoustic pressure field prediction program, and a method for implementing acoustic pressure field generation artificial intelligence, and more particularly, to a transcranial focused ultrasound acoustic pressure field prediction device, a transcranial focused ultrasound acoustic pressure field prediction program, and a method for implementing acoustic pressure field generation artificial intelligence, capable of rapidly predicting a shape of an ultrasound acoustic pressure field formed in a transcranial region according to a given shape of a skull and a position of an ultrasonic transducer.

### BACKGROUMD ART

Since focused ultrasound (FUS) may perform a medical treatment noninvasively by emitting acoustic energy concentrated in a local region within a biological tissue, the focused ultrasound is used for treatments in various regions. For a noninvasive treatment using the focused ultrasound, the ultrasound has to be capable of being emitted to a desired region. However, the ultrasound is invisible, and exhibits reflection and refraction characteristics when transmitted to a tissue in a living body.

In order to solve the above problem, a magnetic-resonance-guided focused ultrasound (MRgFUS) system for identifying a temperature variation through magnetic resonance to visualize ultrasound has been developed. However, the MRgFUS system has a problem that a procedure requires a long time. In addition, in a case of a transcranial treatment, low-intensity focused ultrasound is mainly used, and it is difficult to use the magnetic-resonance-guided focused ultrasound system because a temperature variation is relatively small in the low-intensity focused ultrasound.

In addition, there is a system for displaying a focal point position of an ultrasonic transducer on a medical image that is acquired in advance by using a real-time optical tracking device through image-guided focused ultrasound (neuro-navigation). However, the system has a limitation that an effect of variations in the focal point position and intensity caused by a skull may not be taken into consideration.

### DISCLOSURE

### Technical Problem

An object of the present invention is to provide a transcranial focused ultrasound acoustic pressure field prediction device, a transcranial focused ultrasound acoustic pressure field prediction program, and a method for implementing acoustic pressure field generation artificial intelligence, capable of rapidly predicting a shape of an ultrasound acoustic pressure field formed in a transcranial region according to a given shape of a skull and a position of an ultrasonic transducer.

In addition, an object of the present invention is to provide a transcranial focused ultrasound acoustic pressure field prediction device, a transcranial focused ultrasound acoustic pressure field prediction program, and a method for implementing acoustic pressure field generation artificial intelligence, capable of predicting an ultrasound acoustic pressure field by reflecting refraction that occurs when ultrasound passes through a skull.

In addition, an object of the present invention is to provide a transcranial focused ultrasound acoustic pressure field prediction device, a transcranial focused ultrasound acoustic pressure field prediction program, and a method for implementing acoustic pressure field generation artificial intelligence, capable of predicting an ultrasound acoustic pressure field at a speed close to a real time with high accuracy.

### Technical Solution

According to one aspect of the present invention, there is provided a transcranial focused ultrasound acoustic pressure field prediction device including: an input/output unit for allowing a user to input data, and outputting the data in a form that is recognizable by the user, a memory for storing a transcranial focused ultrasound acoustic pressure field prediction program; and a control unit for executing the transcranial focused ultrasound acoustic pressure field prediction program to derive result data according to the data input through the input/output unit, wherein the control unit outputs ultrasound acoustic pressure field data formed in a transcranial region by ultrasound applied by an ultrasonic transducer when shape data of a skull and position data of the ultrasonic transducer are input.

In addition, the control unit may output the ultrasound acoustic pressure field data formed by the ultrasound applied by the ultrasonic transducer to the shape data of the skull by reflecting that refraction occurs as the ultrasound passes through the skull.

In addition, the position data of the ultrasonic transducer may include coordinate data representing three-dimensional coordinates at which the ultrasonic transducer is positioned with respect to the skull, and angle data representing an angle at which the ultrasonic transducer is positioned with respect to the skull.

In addition, the transcranial focused ultrasound acoustic pressure field prediction program may be provided by artificial intelligence based on a deep neural network (DNN).

In addition, the transcranial focused ultrasound acoustic pressure field prediction program may include: an encoder for training a sparse matrix for the shape data of the skull, which is compressed; and a decoder for receiving the sparse matrix for the skull and the position data of the ultrasonic transducer, which is normalized, to generate an ultrasound acoustic pressure field in a region of interest corresponding to the received sparse matrix and the received position data.

In addition, the transcranial focused ultrasound acoustic pressure field prediction program may be configured based on an autoencoder model based on a convolutional neural network.

In addition, the transcranial focused ultrasound acoustic pressure field prediction program may be configured based on a pix2pix model.

In addition, the transcranial focused ultrasound acoustic pressure field prediction program may be configured based on a variational autoencoder model.

In addition, the artificial intelligence may use training data including the shape data of the skull, the position data of the ultrasonic transducer, and the ultrasound acoustic pressure field data to perform training so as to predict a shape of an ultrasound acoustic pressure field according to the shape data of the skull and the position data of the ultrasonic transducer by using the shape data of the skull and the position data of the ultrasonic transducer as inputs and using the ultrasound acoustic pressure field data corresponding to the inputs as an output.

According to another aspect of the present invention, there is provided a transcranial focused ultrasound acoustic pressure field prediction program, wherein the transcranial focused ultrasound acoustic pressure field prediction program is stored in a recording medium that is readable by a transcranial focused ultrasound acoustic pressure field prediction device in order to allow the transcranial focused ultrasound acoustic pressure field prediction device to execute: inputting shape data of a skull and position data of an ultrasonic transducer; and outputting ultrasound acoustic pressure field data formed in a transcranial region by ultrasound applied by the ultrasonic transducer.

In addition, the position data of the ultrasonic transducer may include coordinate data representing three-dimensional coordinates at which the ultrasonic transducer is positioned with respect to the skull, and angle data representing an angle at which the ultrasonic transducer is positioned with respect to the skull.

According to still another aspect of the present invention, there is provided a method for implementing acoustic pressure field generation artificial intelligence, which is provided by a transcranial focused ultrasound acoustic pressure field prediction program stored in a recording medium that is readable by a transcranial focused ultrasound acoustic pressure field prediction device, the method including: preparing training data including shape data of a skull, position data of an ultrasonic transducer, and ultrasound acoustic pressure field data; and allowing artificial intelligence to perform training so as to predict an ultrasound acoustic pressure field formed in a transcranial region by ultrasound applied by the ultrasonic transducer by using the shape data of the skull and the position data of the ultrasonic transducer as inputs and using the ultrasound acoustic pressure field data corresponding to the inputs as an output in the training data.

### Advantageous Effects

According to one embodiment of the present invention, a transcranial focused ultrasound acoustic pressure field prediction device, a transcranial focused ultrasound acoustic pressure field prediction program, and a method for implementing acoustic pressure field generation artificial intelligence, capable of rapidly predicting a shape of an ultrasound acoustic pressure field formed in a transcranial region according to a given shape of a skull and a position of an ultrasonic transducer, can be provided.

In addition, according to one embodiment of the present invention, a transcranial focused ultrasound acoustic pressure field prediction device, a transcranial focused ultrasound acoustic pressure field prediction program, and a method for implementing acoustic pressure field generation artificial intelligence, capable of predicting an ultrasound acoustic pressure field by reflecting refraction that occurs when ultrasound passes through a skull, can be provided.

In addition, according to one embodiment of the present invention, a transcranial focused ultrasound acoustic pressure field prediction device, a transcranial focused ultrasound acoustic pressure field prediction program, and a method for implementing acoustic pressure field generation artificial intelligence, capable of predicting an ultrasound acoustic pressure field at a speed close to a real time with high accuracy, can be provided.

### DESCRIPTION OF DRAWINGS

FIG. 1 is a view showing a transcranial focused ultrasound acoustic pressure field prediction device according to one embodiment of the present invention.

FIG. 2 is a view showing a process of deriving result data by the transcranial focused ultrasound acoustic pressure field prediction device according to one embodiment of the present invention.

FIG. 3 is a flow chart showing a method for allowing acoustic pressure field generation artificial intelligence provided by a transcranial focused ultrasound acoustic pressure field prediction program to perform training according to one embodiment of the present invention.

FIGS. 4 and 5 are views showing a configuration of data to acquire training data by performing a simulation based on computational mechanics.

FIG. 6 is a block diagram showing an operation process of the acoustic pressure field generation artificial intelligence.

FIG. 7 is a view showing a structure of acoustic pressure field generation artificial intelligence according to a first embodiment of the present invention.

FIG. 8 is a view showing a structure of acoustic pressure field generation artificial intelligence according to a second embodiment of the present invention.

FIG. 9 is a view showing a structure of acoustic pressure field generation artificial intelligence according to a third embodiment of the present invention.

FIG. 10 is a view for describing a distance between focal point coordinates of an ultrasound acoustic pressure field predicted by the acoustic pressure field generation artificial intelligence, which is subjected to the training, and focal point coordinates corresponding to a correct answer.

FIG. 11 is a view for describing a Dice similarity coefficient.

FIG. 12 is a view for describing a Hausdorff distance.

FIG. 13 is a view that visualizes the ultrasound acoustic pressure field predicted by the acoustic pressure field generation artificial intelligence according to one embodiment of the present invention, an ultrasound acoustic pressure field predicted by a computational analysis-based simulation, and an error between focal point regions to compare the ultrasound acoustic pressure fields with each other.

FIG. 14 is a view for describing a water tank experiment for measuring an acoustic pressure field of ultrasound in an actual space.

FIG. 15 is a view that compares the ultrasound acoustic pressure field predicted by the acoustic pressure field generation artificial intelligence according to one embodiment of the present invention with an ultrasound acoustic pressure field measured from the water tank experiment.

### MODE FOR INVENTION

Hereinafter, exemplary embodiments of the present invention will be described in detail with reference to the accompanying drawings. However, the technical idea of the present invention is not limited to the embodiments described herein, but may be realized in different forms. The embodiments introduced herein are provided to sufficiently deliver the idea of the present invention to those skilled in the art so that the disclosed contents may become thorough and complete.

When it is mentioned in the present disclosure that one element is on another element, it means that one element may be directly formed on another element, or a third element may be interposed between one element and another element. Further, in the drawings, thicknesses of films and regions are exaggerated for effective description of the technical contents.

In addition, in various embodiments of the present disclosure, the terms such as first, second, and third are used to describe various elements, but the elements are not limited by the terms. The terms are used only to distinguish one element from another element. Therefore, an element mentioned as a first element in one embodiment may be mentioned as a second element in another embodiment. The embodiments described and illustrated herein include their complementary embodiments. Further, the term “and/or” used herein is used to include at least one of the elements enumerated before and after the term.

As used herein, expressions in a singular form include a meaning of a plural form unless the context clearly indicates otherwise. Further, the terms such as “including” and “having” are intended to designate the presence of features, numbers, steps, elements, or combinations thereof described in the present disclosure, and shall not be construed to preclude any possibility of the presence or addition of one or more other features, numbers, steps, elements, or combinations thereof. In addition, the term “connection” used herein is used to include both indirect and direct connections of a plurality of elements.

Further, in the following description of the present invention, detailed descriptions of known functions or configurations incorporated herein will be omitted when they may make the gist of the present invention unnecessarily unclear.

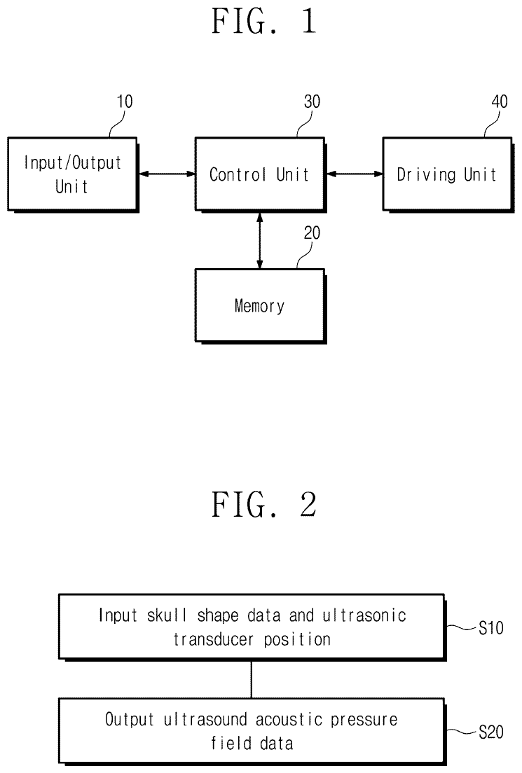

FIG. 1 is a view showing a transcranial focused ultrasound acoustic pressure field prediction device according to one embodiment of the present invention.

Referring to FIG. 1, according to one embodiment of the present invention, a transcranial focused ultrasound acoustic pressure field prediction device may include an input/output unit 10, a memory 20, a control unit 30, and a driving unit 40.

The input/output unit 10 may allow a user to input data, and output the data in a form that is recognizable by the user. For example, the input/output unit 10 may be provided as a keyboard, a mouse, a digitizing pad, or the like for allowing a user to input data. In addition, the input/output unit 10 may include a display panel and the like for outputting the data. In addition, the input/output unit 10 may be provided as a touch screen in which an input portion and an output portion are integrated. In addition, the input/output unit 10 may be provided in a form in which a portion for inputting data or a portion for outputting data is integrated with another medical device, so that the medical device may input the data or output the data.

The memory 20 may store the data. The memory 20 may be provided such that a transcranial focused ultrasound acoustic pressure field prediction program is stored therein. In this case, the transcranial focused ultrasound acoustic pressure field prediction program may be implemented based on artificial intelligence. In detail, the transcranial focused ultrasound acoustic pressure field prediction program may be implemented by artificial intelligence based on a deep neural network (DNN). In addition, the memory 20 may store the data input through the input/output unit 10 and data generated by applying the data input through the input/output unit 10 to the transcranial focused ultrasound acoustic pressure field prediction program.

The control unit 30 may execute the transcranial focused ultrasound acoustic pressure field prediction program stored in the memory 20 to derive result data according to the data input through the input/output unit 10 and output the derived result data through the input/output unit 10.

The driving unit 40 may control an operation state of an ultrasonic transducer for generating focused ultrasound. The driving unit 40 may control a position of the ultrasonic transducer according to an ultrasound acoustic pressure field predicted by the control unit 30.

FIG. 2 is a view showing a process of deriving result data by the transcranial focused ultrasound acoustic pressure field prediction device according to one embodiment of the present invention.

Referring to FIG. 2, according to one embodiment of the present invention, the transcranial focused ultrasound acoustic pressure field prediction device may, when shape data of a skull and position data of an ultrasonic transducer are input (S10), output ultrasound acoustic pressure field data formed in a transcranial region by ultrasound applied by the input ultrasonic transducer (S20). The position data of the ultrasonic transducer may be provided as position data on an outer side of a head of a living body. The ultrasound acoustic pressure field data may be set as a three-dimensional position and a shape of an acoustic pressure field formed by the ultrasound in a region where a brain is positioned inside the skull in the living body. Accordingly, when the position of the ultrasonic transducer is input for a specific skull shape, the ultrasound acoustic pressure field data formed inside the skull may be output and recognized in real time.

Focused ultrasound (FUS) may emit acoustic energy concentrated in a local region within a biological tissue. Accordingly, the focused ultrasound has been used for purposes such as diagnosis and a treatment by applying energy to an inside of a human body. In particular, it has been confirmed that the focused ultrasound may stimulate the brain noninvasively when applied to the brain, so that the focused ultrasound may be used for brain stimulation and treatments.

The focused ultrasound may be divided into high-intensity focused ultrasound (HIFU) and low-intensity focused ultrasound (LIFU) depending on intensity. Similar to thrombolysis, extracorporeal shockwaves, thermal ablation, boiling histotripsy, and the like, the high-intensity focused ultrasound may directly change a condition of a target region to produce a treatment effect.

Meanwhile, the low-intensity focused ultrasound may be used in fields such as drug delivery through opening of a blood-brain barrier and noninvasive brain stimulation. In addition, the low-intensity focused ultrasound has recently been found to be effective in treating neurological diseases such as epilepsy, brain tumors, an Alzheimer's disease, and a Parkinson's disease.

In order for a treatment with the focused ultrasound to be effective, the ultrasound has to be capable of being emitted to a desired region. However, the ultrasound is invisible, and exhibits reflection and refraction characteristics when traveling in the living body. In particular, the ultrasound may have distortion when passing through a boundary at which regions with mutually different physical properties meet due to wave characteristics, so that severe distortion may occur when the ultrasound passes through the skull or a porous region. Accordingly, it is difficult to use the ultrasound for a transcranial treatment.

Meanwhile, according to one embodiment of the present invention, the transcranial focused ultrasound acoustic pressure field prediction device may provide the ultrasound acoustic pressure field data formed in an inside of the transcranial region when the ultrasonic transducer is positioned on the outer side of the skull corresponding to the input shape data of the skull, by reflecting that refraction occurs as the ultrasound travels and passes through regions including the skull. Accordingly, the user who performs a medical practice may effectively perform the medical practice using the ultrasound through the ultrasound acoustic pressure field data provided by the transcranial focused ultrasound acoustic pressure field prediction device.

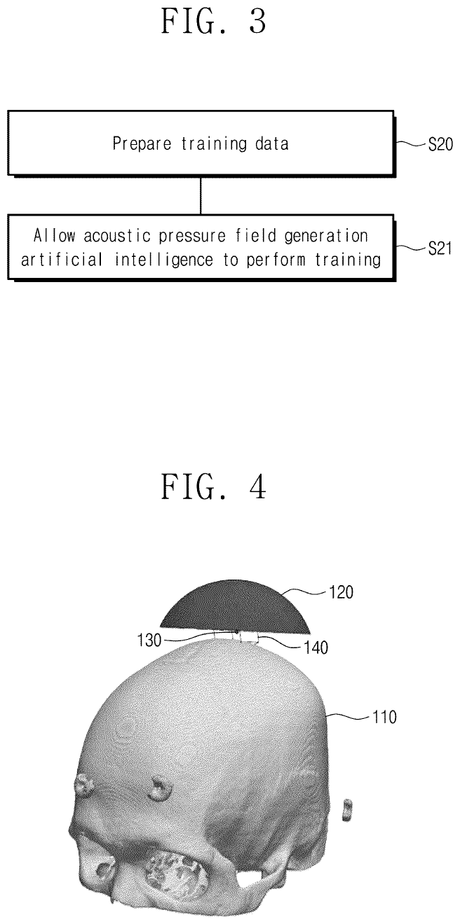

FIG. 3 is a flow chart showing a method for allowing acoustic pressure field generation artificial intelligence provided by a transcranial focused ultrasound acoustic pressure field prediction program to perform training according to one embodiment of the present invention.

Referring to FIG. 3, training data for allowing acoustic pressure field generation artificial intelligence to perform training may be prepared (S20). The training data may include shape data of a skull, position data of an ultrasonic transducer, and acoustic pressure field shape data.

The shape data of the skull may be data on a three-dimensional shape of the skull. The shape data of the skull may be provided in the form of a three-dimensional image of the skull. The shape data of the skull may be an image taken through computed tomography (CT) scanning or the like.

The position data of the ultrasonic transducer may be data on a point at which the ultrasonic transducer is positioned with respect to the skull. The position data of the ultrasonic transducer may include coordinate data representing three-dimensional coordinates at which the ultrasonic transducer is positioned with respect to the skull, and angle data representing an angle at which the ultrasonic transducer is positioned with respect to the skull (i.e., an angle at which the ultrasound is emitted with respect to the skull).

The acoustic pressure field shape data may be data on a shape in which the ultrasound emitted from the ultrasonic transducer travels inside the skull, that is, in a region where the brain is positioned. The acoustic pressure field shape data may be provided in the form of a three-dimensional image, a two-dimensional image for a specific reference plane, or the like. The acoustic pressure field shape data may be paired with the shape data of the skull and the position data of the ultrasonic transducer that applies the ultrasound to the skull. The acoustic pressure field shape data may be binarized into a focal point region and a focal point outside region of the ultrasound through a full-width-at-half-maximum (FWHM) threshold according to a definition of a focal point of the focused ultrasound, as will be described below. In addition, a boundary of the focal point region may be converted from a boundary based on the FWHM threshold into an ellipsoid boundary.

The training data may be acquired by positioning the ultrasonic transducer to be adjacent to the skull of the living body, and photographing a shape of the skull and a path in which the ultrasound travels through an existing medical device while emitting the ultrasound.

In addition, the training data may be acquired through a simulation based on computational mechanics.

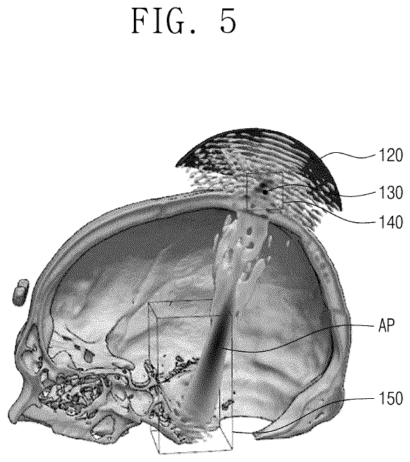

FIGS. 4 and 5 are views showing a configuration of data to acquire training data by performing a simulation based on computational mechanics.

Referring to FIGS. 4 and 5, data for numerical modeling of a simulation may include a skull model 110, an ultrasonic transducer model 120, and a region of interest 150.

The skull model 110 may correspond to the shape data of the skull of the training data. The skull model 110 may be provided by photographing an actual skull. For example, the skull model 110 may be provided by performing CT scanning on the actual skull. The skull model 110 may be provided with a set spatial resolution or more to have satisfactory precision and a satisfactory spatial resolution in water. For example, the skull model 110 may be provided by performing the CT scanning to have a spatial resolution of 0.5 mm×0.5 mm×0.5 mm or more. Voxels of the skull model 110 may be classified into water, a cancellous bone, and a cortical bone according to Hounsfield units, as shown in Mathematical Formula 1.

$${c}_{i , j , k} = \{ \begin{matrix}1500 m / s , & for {ϕ}_{i , j , k} \leq 0 \\ 2140 m / s , & for 0 < {ϕ}_{i , j , k} \leq 1000 \\ 2384 m / s , & for 1000 < {ϕ}_{i , j , k}\end{matrix} \qquad [ Formula 1 ]$$

$${ρ}_{i , j , k} = \{ \begin{matrix}1000 kg / {m}^{3} , & for {ϕ}_{i , j , k} \leq 0 \\ 1000 + 1.19 {ϕ}_{i , j , k} kg / {m}^{3} , & for 0 < {ϕ}_{i , j , k} \leq 1000 \\ 2190 kg / {m}^{3} , & for 1000 < {ϕ}_{i , j , k}\end{matrix}$$

$${a}_{i , j , k} = 33 Np / m , \qquad for 0 < {ϕ}_{i , j , k}$$

c_{i,j,k}

is a velocity of ultrasound, and

ρ_{i,j,k}

is a density of ultrasound. In this case,

ϕ_{i,j,k}≤0

is water,

0<ϕ_{i,j,k}≤1000

is a cancellous bone, and

1000<ϕ_{i,j,k}

is a cortical bone.

α_{i,j,k}

is an attenuation coefficient.

A plurality of skull models 110 may be created by using at least two different actual skulls, respectively.

The ultrasonic transducer model 120 may be acquired by modeling an actual ultrasonic transducer. The ultrasonic transducer model 120 may be modeled to have a preset diameter, a preset radius of curvature, and a preset focal length. For example, the ultrasonic transducer model 120 may be modeled to have a diameter of 96 mm, a radius of curvature of 52 mm, and a focal length of 83 mm. A position reference point 130 may be set in the ultrasonic transducer model 120, so that coordinates of the position reference point may be set as a position of the ultrasonic transducer model 120. For example, a central region of an exit surface facing the skull model 110 in the ultrasonic transducer model 120 may be set as the position reference point 130. Simulations may be performed while the position of the ultrasonic transducer model 120 varies in a movement region 140 that is adjacent to the skull model 110. The movement region 140 may be adjacent to an upper outer surface of the skull model 110, and may be set to have a preset volume. For example, the movement region 140 may be set to have a size of 20 mm×20 mm×20 mm.

The position of the ultrasonic transducer model 120 may include simulation coordinate data representing three-dimensional coordinates of the position reference point 130, and simulation angle data representing an angle at which the ultrasonic transducer model 120 is positioned with respect to the skull model 110. The simulation angle data may be defined as a normal vector of the exit surface. The simulation coordinate data and the simulation angle data may correspond to the coordinate data and the angle data of the ultrasonic transducer, respectively.

The region of interest 150 may be a region for acquiring data on a shape in which the ultrasound emitted from the ultrasonic transducer model 120 propagates, and may be set to have a preset volume. For example, the region of interest 150 may be set to have a size of 40 mm×75 m×40 mm.

A center point of the region of interest 150 may be set at a position spaced apart from an initial position of the ultrasonic transducer model 120 by the focal length. A center of a plane in which the region of interest starts (x=20 mm, y=0 mm, z=20 mm) may be set to have a distance of 50 mm from center coordinates of the exit surface of the ultrasonic transducer. A simulation for acquiring the shape in which the ultrasound propagates in the region of interest 150 may be performed while varying the position of the ultrasonic transducer model 120. In order to perform ultrasonic propagation modeling in the region of interest 150, the Westervelt-Lighthill equation as shown in [Mathematical Formula 2] may be used as a governing equation.

$${∇}^{2} p - \frac{1}{{c}^{2}} \frac{{\partial }^{2} p}{\partial {t}^{2}} - \frac{\tilde{a}}{{c}^{2}} \frac{\partial p}{\partial t} = 0 with \tilde{a} = 2 a \sqrt{\frac{{a}^{2} {c}^{4}}{4 {π}^{2} {f}^{2}} + {c}^{2}} \qquad [ Formula 2 ]$$

In this case, p is a pressure of sound, c is a wave velocity in a medium, a is an attenuation coefficient in a medium, f is a frequency of the sound, and t is a time.

A finite-difference time-domain (FDTD) method as shown in [Mathematical Formula 3] may be used to approximate partial derivatives of the governing equation with respect to a space and a time, and ultrasonic propagation may be performed by repeatedly evaluating the Formula.

$${}_{}^{t + Δ t} {P}_{i , j , k}^{} = {}_{}^{t} {P}_{i , j , k}^{} - {C}_{i , j , k}^{p} [ ( {}_{}^{t + 1 / 2 Δ t} {V}_{i , j , k}^{x} - {}_{}^{t + 1 / 2 Δ t} {V}_{i - 1 , j , k}^{x} ) + ( {}_{}^{t + 1 / 2 Δ t} {V}_{i , j , k}^{y} - {}_{}^{t + 1 / 2 Δ t} {V}_{i , j - 1 , k}^{y} ) + ( {}_{}^{t + 1 / 2 Δ t} {V}_{i , j , k}^{z} - {}_{}^{t + 1 / 2 Δ t} {V}_{i , j , k - 1}^{z} ) ] - {A}_{i , j , k} {}_{}^{t} {P}_{i , j , k}^{} , \qquad [ Formula 3 ]$$

$${}_{}^{t + 1 / 2 Δ t} {V}_{i , j , k}^{x} = {}_{}^{t - 1 / 2 Δ t} {V}_{i , j , k}^{x} - {C}_{i , j , k}^{vx} [ {}_{}^{t} {P}_{i + 1 , j , k}^{} - {}_{}^{t} {P}_{i , j , k}^{} ] ,$$

$${}_{}^{t + 1 / 2 Δ t} {V}_{i , j , k}^{y} = {}_{}^{t - 1 / 2 Δ t} {V}_{i , j , k}^{y} - {C}_{i , j , k}^{vy} [ {}_{}^{t} {P}_{i , j + 1 , k}^{} - {}_{}^{t} {P}_{i , j , k}^{} ] ,$$

$${}_{}^{t + 1 / 2 Δ t} {V}_{i , j , k}^{z} = {}_{}^{t - 1 / 2 Δ t} {V}_{i , j , k}^{z} - {C}_{i , j , k}^{vz} [ {}_{}^{t} {P}_{1 , j , k + 1}^{} - {}_{}^{t} {P}_{i , j , k}^{} ] ,$$

$${C}_{i , j , k}^{p} = {ρ}_{i , j , k} {c}_{i , j , k}^{2} \frac{Δ t}{δ} , {A}_{i , j , k} = 2 Δ {ta}_{i , j , k} \sqrt{\frac{{a}_{i , j , k}^{2} {c}_{i , j , k}^{4}}{4 {π}^{2} {f}^{2}} + {c}_{i , j , k}^{2}} ,$$

$${C}_{i , j , k}^{v x} = \frac{2}{{ρ}_{i , j , k} + {ρ}_{i + 1 , j , k}} \frac{Δ t}{δ} ,$$

$${C}_{i , j , k}^{vy} = \frac{2}{{ρ}_{i , j , k} + {ρ}_{i , j + 1 , k}} \frac{Δ t}{δ} ,$$

$${C}_{i , j , k}^{vz} = \frac{2}{{ρ}_{i , j , k} + {ρ}_{i , j , k + 1}} \frac{Δ t}{δ} .$$

In this case,
^{t}P_{i,j,k}

is a pressure of sound at a nodal point (acoustic pressure value),
^{t}V_{i,j,k}^{x},
^{t}V_{i,j,k}^{y}, and
^{t}V_{i,j,k}^{z}

are velocities of sound on x-, y-, and z-axes at a time t at a nodal point, respectively,

c_{i,j,k}

is a velocity of ultrasound,

ρ_{i,j,k}

is a density of ultrasound, and

α_{i,j,k}

is an attenuation coefficient.

In this case, since an algorithm for solving explicit dynamic processes, including PDTD, has numerical errors, a stability condition known as the Courant-Friedrichs-Lewy (CFL) criterion, which may be expressed as [Mathematical Formula 4], has to be satisfied.

$$Δ t \leq \frac{δ}{\sqrt{3} c} \qquad [ Formula 4 ]$$

In this case,

Δt

is a time interval,

δ

is a discretized spatial interval, and c is a velocity of a wave.

An appropriate frequency of the focused ultrasound used in the transcranial region has been known to be less than 650 kHz. In the present embodiment, ultrasound of 250 kHz was emitted to perform the numerical modeling and perform the simulation for acquiring the training data.

When the simulation based on computational mechanics is performed, a pressure distribution of the ultrasound in the region of interest according to the position of the ultrasonic transducer model 120 may be acquired as the acoustic pressure field shape data. In this case, simulations may be performed on skull models 110 having mutually different shapes, respectively, to acquire results thereof.

The simulation results of an acoustic pressure field shape may be binarized into the focal point region and the focal point outside region of the ultrasound through the definition of the focal point of the focused ultrasound. The focal point region of the focused ultrasound may be defined as a region corresponding to the full-width-at-half-maximum (FWHM) threshold.

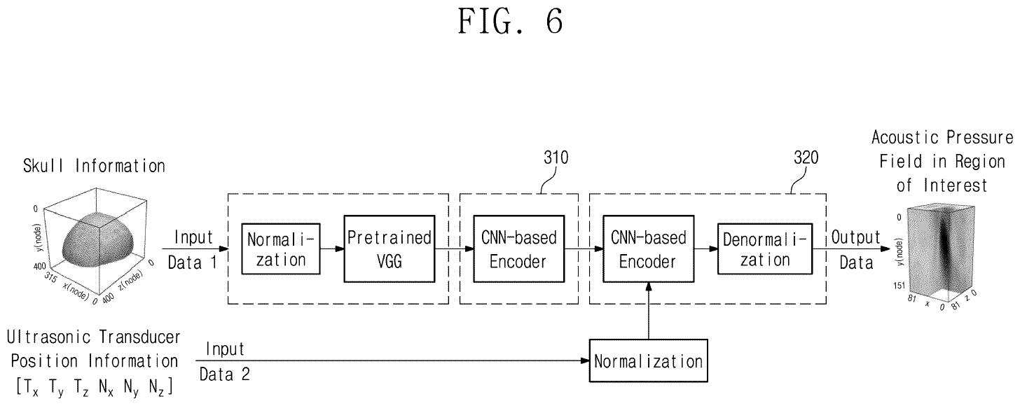

FIG. 6 is a block diagram showing an operation process of the acoustic pressure field generation artificial intelligence.

Referring to FIG. 6, the acoustic pressure field generation artificial intelligence may train a relation between the shape data of the skull and the position of the ultrasonic transducer, and an ultrasound acoustic pressure field generated according to the shape data of the skull and the position of the ultrasonic transducer, through the prepared training data (S21).

Thereafter, the acoustic pressure field generation artificial intelligence may output the ultrasound acoustic pressure field data that will be generated according to the shape data of the skull and the position of the ultrasonic transducer when the shape data of the skull and the position of the ultrasonic transducer are input.

In detail, the acoustic pressure field generation artificial intelligence may be subjected to the training through the prepared training data.

The training data may be used to perform the training so as to predict the ultrasound acoustic pressure field data according to a shape of the skull and the positon of the ultrasonic transducer by using the shape data of the skull and position data of the ultrasonic transducer as inputs and using the ultrasound acoustic pressure field data formed in a region of interest according to the inputs as an output.

For efficient training of the acoustic pressure field generation artificial intelligence, the shape data of the skull may be dimensionally compressed through a visual geometry group (VGG) network that is pretrained with ImageNet, which is a large-scale visual database. For example, after the shape data of the skull is normalized, the shape data of the skull may be dimensionally compressed into voxel data having a size of 315*512 by using a VGG19 network. In this case, the VGG19 network, which is pretrained, may perform the dimensional compression by adding a global average pooling layer to a feature extraction unit including a convolutional layer to receive data, which has a voxel size that is different from a voxel size of image data used in previous training, as an input value.

The position data of the ultrasonic transducer may include three-dimensional coordinate data and angle data. In this case, for the efficient training of the artificial intelligence, the coordinate data and the angle data may be converted into values between −1 and 1 through a minimum-maximum normalization process, respectively. The artificial intelligence may train through error backpropagation.

Regarding the acoustic pressure field shape data, a part set as the focal point region may be used as an output. In this case, the focal point region may be transformed into a boundary according to the FWHM threshold. During a process of the training, the acoustic pressure field data may be used in a normalized form. Thereafter, when the acoustic pressure field generation artificial intelligence primarily outputs the ultrasound acoustic pressure field data according to the shape data of the skull and the position of the ultrasonic transducer, a primary output value may be denormalized, and final ultrasound acoustic pressure field data may be output.

The acoustic pressure field generation artificial intelligence may be configured based on a convolutional neural network (CNN). The acoustic pressure field generation artificial intelligence may include: an encoder 310 for training a sparse matrix for the shape data of the skull, which is compressed; and a decoder 320 for receiving the sparse matrix for the skull and the position data of the ultrasonic transducer, which is normalized, to generate an ultrasound acoustic pressure field in a region of interest corresponding to the received sparse matrix and the received position data.

The shape data of the skull may be input to the encoder 310. An output of the encoder 310 and the position data of the ultrasonic transducer may be input to the decoder 320 together.

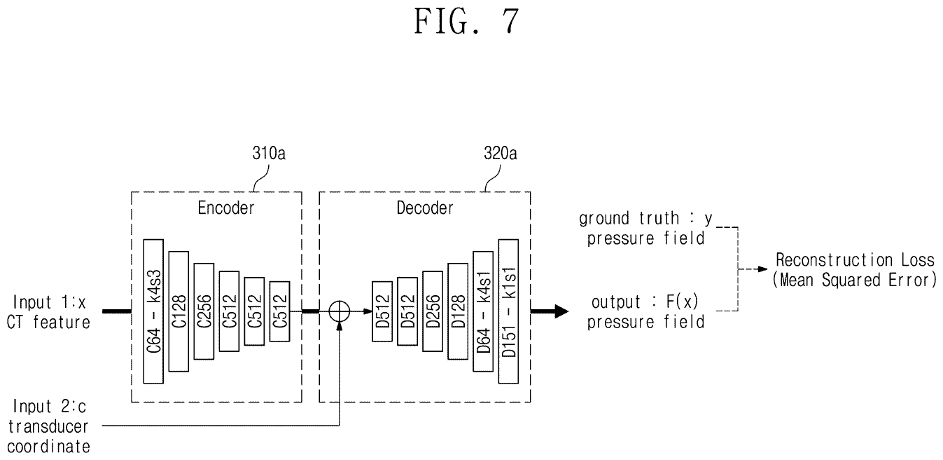

FIG. 7 is a view showing a structure of acoustic pressure field generation artificial intelligence according to a first embodiment of the present invention.

Referring to FIG. 7, acoustic pressure field generation artificial intelligence according to a first embodiment of the present invention may be configured based on an autoencoder (AE) model based on a convolutional neural network.

The encoder 310a may include six convolutional layers. The decoder 320a may include six transposed convolutional layers. The encoder 310a may generate 512 sparse matrices from the compressed shape data of the skull, and the decoder 320a may receive the sparse matrices for the skull input from the encoder and the position data of the ultrasonic transducer together to convert the received sparse matrices and the received position data into the ultrasound acoustic pressure field. In this case, C may represent the number of feature maps in each of the convolutional layers in the encoder 310a. An input layer of the encoder 310a may be provided with 64 feature maps C, a kernel size k of 4, and a stride s of 3. The encoder 310a may use batch normalization, and a transfer function may be LeakyReLU. A first layer of the encoder 310a may have 64 feature maps C, a kernel size k of 4, and a stride s of 3. A second layer of the encoder 310a may have 128 feature maps C. A third layer of the encoder 310a may have 256 feature maps C. Each of fourth to sixth layers of the encoder 310a may have 512 feature maps C.

In each of layers of the decoder 320a, D may represent the number of feature maps in the transposed convolutional layer, k may represent a kernel size, s may represent a stride, and F may represent the number of nodes in a fully connected layer. The decoder 320a may use batch normalization, and a transfer function may be ReLU. Each of first and second layers of the decoder 320a may have 512 feature maps D. A third layer of the decoder 320a may have 256 feature maps D. A fourth layer of the decoder 320a may have 128 feature maps D. A fifth layer of the decoder 320a may have 64 feature maps D, a kernel size k of 4, and a stride s of 1. A sixth layer of the decoder 320a may have 151 feature maps D, a kernel size k of 4, and a stride s of 1.

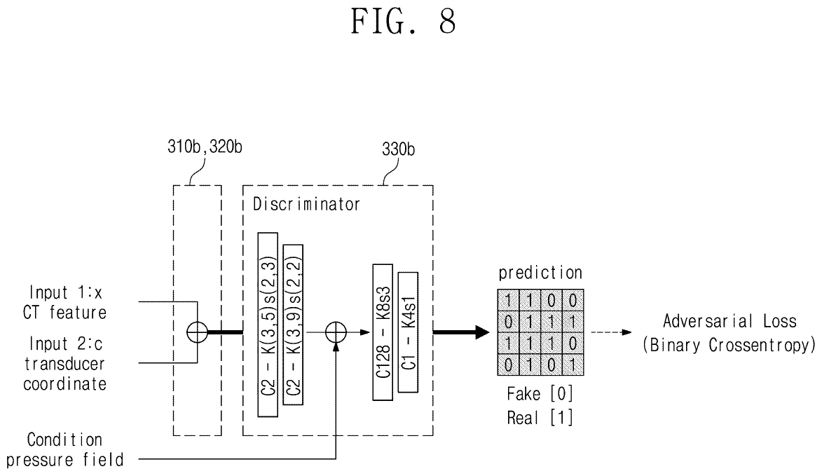

FIG. 8 is a view showing a structure of acoustic pressure field generation artificial intelligence according to a second embodiment of the present invention.

Referring to FIG. 8, acoustic pressure field generation artificial intelligence according to a second embodiment of the present invention may be configured based on a pix2pix model. The pix2pix model may be a model specialized for image conversion among generative adversarial networks in which generators 310b and 320b and a discriminator 330b perform adversarial training to improve performance of the network based on a convolutional neural network structure.

The generator 310b and 320b may include an encoder 310b and a decoder 320b, and may receive the compressed shape data of the skull and the position of the ultrasonic transducer to generate the ultrasound acoustic pressure field data. Structures of the encoder 310b and the decoder 320b may be identical to the structures of the encoder and decoder of the acoustic pressure field generation artificial intelligence according to the first embodiment of the present invention, respectively.

The discriminator 330b may affect network training by discriminating, based on a patch, whether the received ultrasound acoustic pressure field data corresponds to an ultrasound acoustic pressure field predicted by the generators 310b and 320b or a correct answer generated from a simulation used as the training data. Since the acoustic pressure field generation artificial intelligence according to the second embodiment of the present invention further includes the discriminator 330b, the generators 310b and 320b may be evaluated for the predicted ultrasound acoustic pressure field, so that the training may be performed to generate an acoustic pressure field with better quality. The discriminator 330b may be configured to have two layers on a front side and a rear side based on a point in which a condition pressure field, which is a reference for discrimination, is input. In each of the layers, C may represent the number of feature maps, k may represent a kernel size, and s may represent a stride.

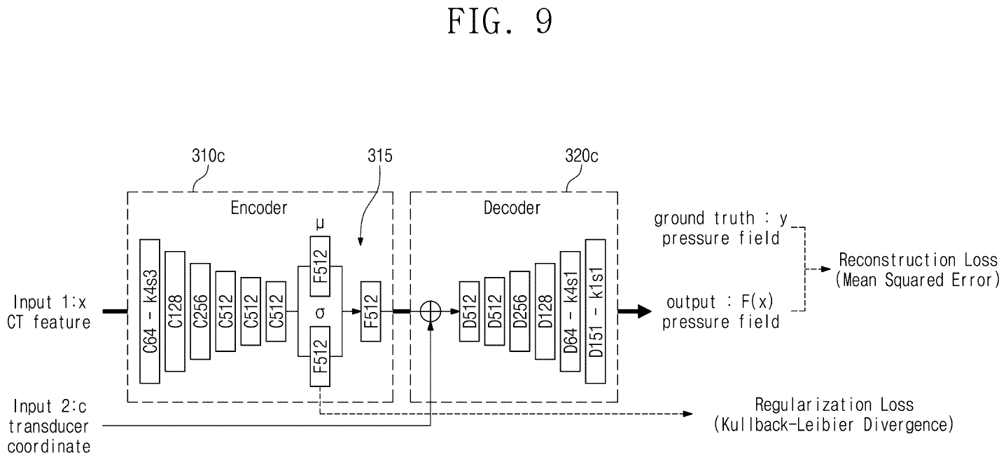

FIG. 9 is a view showing a structure of acoustic pressure field generation artificial intelligence according to a third embodiment of the present invention.

Referring to FIG. 9, acoustic pressure field generation artificial intelligence according to a third embodiment of the present invention may be configured based on a variational autoencoder (VAE) model based on a convolutional neural network structure.

The acoustic pressure field generation artificial intelligence according to the third embodiment of the present invention may further include a bottleneck unit 315 positioned at a point connected to a decoder 320c at a rear end of an encoder 310c. The bottleneck unit 315 may generate a sparse vector for an input value by using a probability distribution, and convert the sparse vector received by the decoder 320c, so that quality of an output generated by the decoder 320c may be improved. In order to control the generated sparse vector, the bottleneck unit 315 may calculate a mean and a variance from the received shape data of the skull, and sample the sparse vector from a normal distribution having a population mean and a population variance corresponding to the calculated mean and the calculated variance, respectively. In the bottleneck unit 315, a dimension of the sparse vector may be set as a hyperparameter. According to one embodiment, the hyperparameter F, which is the dimension of the sparse vector, may be set to 512. A structure of a portion of the encoder 310c positioned on a front side of the bottleneck unit 315 and a structure of the decoder 320c may be identical to the structures of the encoder and decoder of the acoustic pressure field generation artificial intelligence according to the first embodiment, respectively.

### EXPERIMENTAL EXAMPLE

The training was performed on the artificial intelligence having the structure shown in FIGS. 7 to 9.

Hyperparameters for allowing the acoustic pressure field generation artificial intelligence according to the first to third embodiments of the present invention to perform the training may be set as shown in Table 1. The acoustic pressure field generation artificial intelligence according to the first to third embodiments basically uses a mean square error for the ultrasound acoustic pressure field in the region of interest as a loss function for error backpropagation. In addition, the acoustic pressure field generation artificial intelligence according to the second embodiment of the present invention uses an adversarial loss function in which the discriminator 330b discriminates authenticity of the acoustic pressure field predicted by the generators 310b and 320b and a correct answer acoustic pressure field together. In this case, a weight between a reconstruction loss function implemented as a mean square error and an adversarial loss function implemented as a binary cross entropy will be defined as lambda (λ). In the present experimental example, a weight of a reconstruction function was 60.

TABLE 1

| | optimizer | learning rate | batch size | epoch | λ | latent dim. |
|---|---|---|---|---|---|---|
| AE | Adam | 0.002 | 32 | 300 | | |
| pix2pix | Adam | 0.0004 | 8 | 200 | 60 | |
| VAE | Adam | 0.005 | 8 | 100 | | 512 |

The acoustic pressure field generation artificial intelligence according to the third embodiment of the present invention uses the Kullback-Leibler divergence (KLD) as a regularization loss function to set the population mean and the population variance for extracting a latent variable from the bottleneck unit 315 together.

The loss function for the acoustic pressure field generation artificial intelligence according to the first embodiment may be expressed as [Mathematical Formula 5].

$${L}_{AutoEncoder} = \frac{1}{n} {\sum }_{i = 1}^{n} {( {y}_{1} - F ( {x}_{i} , {c}_{i} ) )}^{2} \qquad [ Formula 5 ]$$

In this case, n represents the number of training data, F represents an autoencoder network, x represents skull shape data, c represents position data of an ultrasonic transducer, and y represents correct answer data for an ultrasound acoustic pressure field.

The loss function for the acoustic pressure field generation artificial intelligence according to the second embodiment may be expressed as [Mathematical Formula 6].

$${L}_{generator} = \frac{1}{n} {\sum }_{i = 1}^{n} [ log ( 1 - D ( {x}_{i} , {c}_{i} , G ( {x}_{i} , {c}_{i} ) ) ) + λ {‖ {y}_{i} - G ( {x}_{i} , {c}_{i} ) ‖}_{1} ]$$

$${L}_{discriminator} = \frac{1}{n} {\sum }_{i = 1}^{n} [ log D ( {x}_{i} , {c}_{i} , {y}_{i} ) + log ( 1 - D ( {x}_{i} , {c}_{i} , G ( {x}_{i} , {c}_{i} ) ) ) ]$$

In this case, n represents the number of training data, x represents skull shape data, c represents position data of an ultrasonic transducer, and y represents correct answer data for an ultrasound acoustic pressure field. In addition, G represents a generator (310b, 320b) network, and D represents a discriminator (330b) network.

The loss function for the acoustic pressure field generation artificial intelligence according to the third embodiment may be expressed as [Mathematical Formula 7].

$${L}_{VariationalAE} = \frac{1}{n} {\sum }_{i = 1}^{n} [ {( {y}_{i} - F ( {x}_{i} , {c}_{i} ) )}^{2} + ( {μ}_{i}^{2} + {σ}_{i}^{2} - ln ( {σ}_{i}^{2} ) - 1 ) ] \qquad [ Formula 7 ]$$

In this case, n represents the number of training data, x represents skull shape data, c represents position data of an ultrasonic transducer, and y represents correct answer data for an acoustic pressure field. In addition, u and o represent a mean and a variance of a Gaussian distribution for sampling a latent vector in a bottleneck unit 315, respectively.

In order to evaluate performance of the acoustic pressure field generation artificial intelligence that is subjected to the training, the focal point region of the ultrasound acoustic pressure field defined as a full-width at half-maximum (FWHM) was compared by using the following four indicators.
1) A distance (AFP) between focal point coordinates of the ultrasound acoustic pressure field predicted by the acoustic pressure field generation artificial intelligence that is subjected to the training and focal point coordinates corresponding to a correct answer is measured.
2) A Hausdorff distance (HD) is calculated to measure a maximum error between a boundary of the focal point region of the ultrasound acoustic pressure field predicted by the acoustic pressure field generation artificial intelligence that is subjected to the training and a boundary of the focal point region corresponding to the correct answer.
3) A Dice similarity coefficient (DSC) is calculated to evaluate a similarity between the focal point region of the ultrasound acoustic pressure field predicted by the acoustic pressure field generation artificial intelligence that is subjected to the training and the focal point region corresponding to the correct answer.
4) A training time and an inference time of a model used in the acoustic pressure field generation artificial intelligence are measured to evaluate practicality of the acoustic pressure field generation artificial intelligence that is subjected to the training.

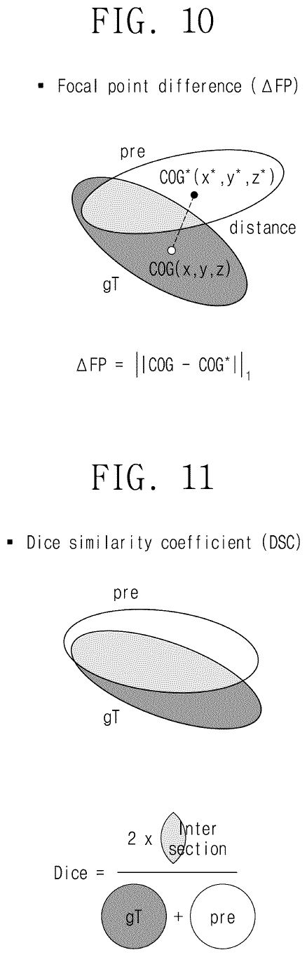

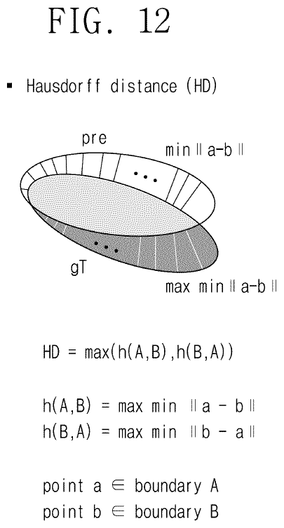

FIG. 10 is a view for describing a distance between focal point coordinates of an ultrasound acoustic pressure field predicted by the acoustic pressure field generation artificial intelligence, which is subjected to the training, and focal point coordinates corresponding to a correct answer, FIG. 11 is a view for describing a Dice similarity coefficient, and FIG. 12 is a view for describing a Hausdorff distance.

In FIGS. 10 to 12, a white region pre represents a focal point region of an ultrasound acoustic pressure field predicted by acoustic pressure field generation artificial intelligence, a dark gray region gT represents a focal point region of an ultrasound acoustic pressure field based on a computational analysis simulation, and a light gray region represents a region in which the two focal point regions coincide with each other.

Table 2 compares the performance of the acoustic pressure field generation artificial intelligence according to the first to third embodiments with each other according to the measurement references described above.

TABLE 2

| | ΔFP [mm] | HD [mm] | DSC (Dice | Training | Test |
|---|---|---|---|---|---|
| | (Focal | (Hausdorff | similarity | time | time |
| | point error) | distance) | coeff.) | [min] | [ms] |
| AE | 5.06 ± 3.62 | 11.81 ± 5.00 | 0.59 ± 0.18 | 439 | 1.44 |
| pix2pix | 4.25 ± 2.70 | 10.34 ± 4.01 | 0.64 ± 0.15 | 1071 | 1.30 |
| VAE | 4.17 ± 2.89 | 10.68 ± 4.19 | 0.64 ± 0.15 | 177 | 1.22 |

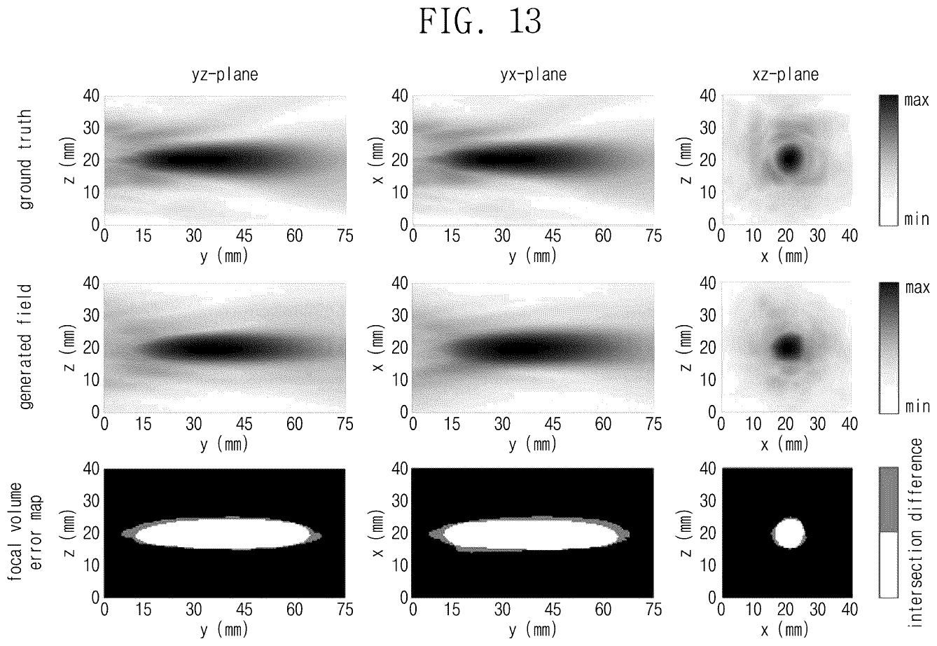

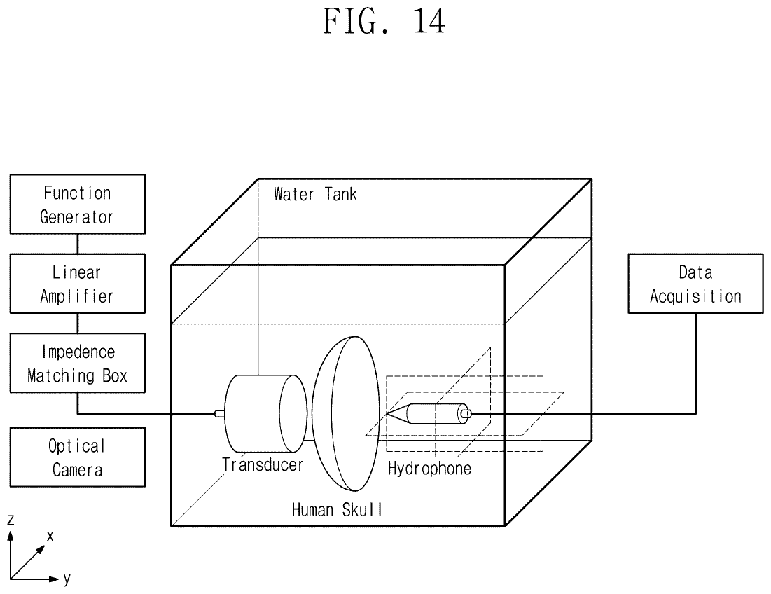

The acoustic pressure field generation artificial intelligence according to the second and third embodiments predicted a position of a focal point of the ultrasound acoustic pressure field to have an average error within 4.25 mm. In this case, it is found that the prediction may be performed such that an average error of the boundary of the focal point region is within 10.7 mm, and a similarity of the focal point region is 0.63 or more. In particular, the acoustic pressure field generation artificial intelligence according to the third embodiment has the shortest training time of an artificial neural network, which is 177 minutes, and also has the shortest prediction time of the ultrasound acoustic pressure field, which is 1.22 ms, so that the acoustic pressure field generation artificial intelligence according to the third embodiment may be evaluated as having the highest practicality among the acoustic pressure field generation artificial intelligence according to the first to third embodiments. FIG. 13 is a view that visualizes the ultrasound acoustic pressure field predicted by the acoustic pressure field generation artificial intelligence according to one embodiment of the present invention, an ultrasound acoustic pressure field predicted by a computational analysis-based simulation, and an error between focal point regions to compare the ultrasound acoustic pressure fields with each other.

It is found that the ultrasound acoustic pressure field predicted by the acoustic pressure field generation artificial intelligence (generated field) and the ultrasound acoustic pressure field predicted by the computational analysis-based simulation (ground truth) have a high morphological similarity. In addition, it is found that the focal point regions defined by the full-width at half-maximum (FWHM) for mutual comparison have a high similarity.

FIG. 14 is a view for describing a water tank experiment for measuring an acoustic pressure field of ultrasound in an actual space.

Referring to FIG. 14, an ultrasonic transducer may be operated such that an electrical signal generated from a function generator and amplified by a linear amplifier is subjected to impedance matching. An actual position and an actual orientation of a skull may be measured by using an optical camera. Four donut-shaped markers may be positioned on the skull, so that coordinate systems in an actual space and a numerical modeling space may be set based on the markers. Acoustic pressure field data for XY, YZ, and XZ planes may be acquired through a hydrophone. A total of 15 experimental data were acquired by performing five experiments and measurements on each of three different skulls, and used to evaluate performance of ultrasound acoustic pressure field prediction data of the acoustic pressure field generation artificial intelligence according to one embodiment of the present invention.

Table 3 compares a value predicted by the acoustic pressure field generation artificial intelligence according to one embodiment of the present invention and the acoustic pressure field data measured by the measurement of the water tank experiment of FIG. 14.

TABLE 3

| | | | |
|---|---|---|---|
| Plane | AE | pix2pix | VAE |

Focal position error (ΔFP) [mm]

| | | | |
|---|---|---|---|
| XY | 3.59 ± 1.03 | 3.96 ± 1.69 | 3.13 ± 1.12 |
| YZ | 3.44 ± 0.72 | 2.97 ± 0.80 | 3.49 ± 0.73 |
| XZ | 0.78 ± 0.29 | 1.03 ± 0.29 | 1.15 ± 0.37 |

Hausdorff distance (HD) [mm]

| | | | |
|---|---|---|---|
| XY | 9.30 ± 2.30 | 8.76 ± 2.55 | 9.34 ± 1.26 |
| YZ | 5.26 ± 0.89 | 4.78 ± 1.00 | 5.72 ± 0.94 |
| XZ | 2.13 ± 0.26 | 2.25 ± 0.14 | 2.29 ± 0.29 |

Dice similarity coefficient (DSC)

| | | | |
|---|---|---|---|
| XY | 0.79 ± 0.04 | 0.79 ± 0.04 | 0.76 ± 0.03 |
| YZ | 0.85 ± 0.03 | 0.86 ± 0.03 | 0.84 ± 0.04 |
| XZ | 0.86 ± 0.02 | 0.83 ± 0.03 | 0.83 ± 0.04 |

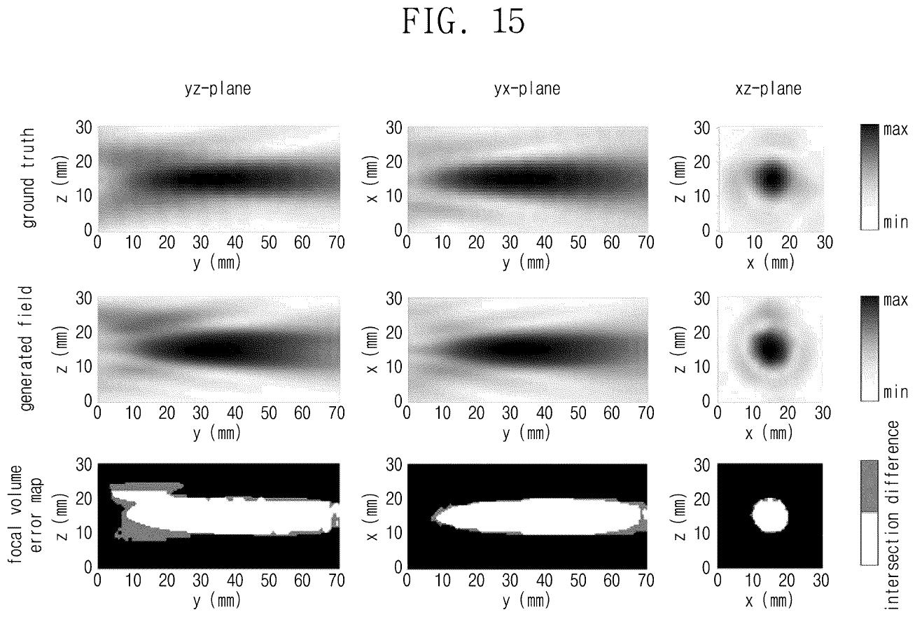

As found through Table 3, the acoustic pressure field generation artificial intelligence according to the first to third embodiments of the present invention exhibit high performance such that an average error in predicting a focal point position of an ultrasound acoustic pressure field in a region of interest is 7.34±2.80 mm, an average error in predicting a boundary of a focal point region is 2.62±0.78 mm, and an average similarity of a focal point region is 0.82±0.03. FIG. 15 is a view that compares the ultrasound acoustic pressure field predicted by the acoustic pressure field generation artificial intelligence according to one embodiment of the present invention with an ultrasound acoustic pressure field measured from the water tank experiment.

It is found that the ultrasound acoustic pressure field predicted by the acoustic pressure field generation artificial intelligence (generated field) and the ultrasound acoustic pressure field measured through the water tank experiment (ground truth) have a high morphological similarity. In addition, it is found that the focal point regions defined by the full-width at half-maximum (FWHM) for mutual comparison have a high similarity.

The transcranial focused ultrasound acoustic pressure field prediction device according to one embodiment of the present invention may calculate a prediction result of an ultrasound acoustic pressure field formed in a transcranial region by an ultrasonic concentrator with high accuracy within a short time. In detail, while an existing computational analysis-based focused ultrasound simulation requires about 100 seconds for each ultrasound acoustic pressure field to calculate the ultrasound acoustic pressure field in the region of interest according to the input of the skull data and the position data of the ultrasonic transducer, it is found that the acoustic pressure field generation artificial intelligence according to one embodiment of the present invention predicts the acoustic pressure field in the region of interest in about 1.2 ms, so that real-time prediction may be performed. In addition, the acoustic pressure field generation artificial intelligence according to one embodiment of the present invention may perform the training based on various skull shape data and require 177 minutes for training of an artificial neural network, so that the acoustic pressure field generation artificial intelligence is highly practical. In addition, data used for the training may be increased to achieve efficient training and performance improvement.

Although the exemplary embodiments of the present invention have been described in detail above, the scope of the present invention is not limited to a specific embodiment, and shall be interpreted by the appended claims. In addition, it is to be understood by those of ordinary skill in the art that various changes and modifications can be made without departing from the scope of the present invention.

1. A transcranial focused ultrasound acoustic pressure field prediction device comprising:
an input/output unit for allowing a user to input data, and outputting the data in a form that is recognizable by the user;
a memory for storing a transcranial focused ultrasound acoustic pressure field prediction program; and
a control unit for executing the transcranial focused ultrasound acoustic pressure field prediction program to derive result data according to the data input through the input/output unit,
wherein the control unit outputs ultrasound acoustic pressure field data formed in a transcranial region by ultrasound applied by an ultrasonic transducer when shape data of a skull and position data of the ultrasonic transducer are input.

2. The transcranial focused ultrasound acoustic pressure field prediction device of claim 1, wherein the control unit outputs the ultrasound acoustic pressure field data formed by the ultrasound applied by the ultrasonic transducer to the shape data of the skull by reflecting that refraction occurs as the ultrasound passes through the skull.

3. The transcranial focused ultrasound acoustic pressure field prediction device of claim 2, wherein the position data of the ultrasonic transducer includes coordinate data representing three-dimensional coordinates at which the ultrasonic transducer is positioned with respect to the skull, and angle data representing an angle at which the ultrasonic transducer is positioned with respect to the skull.

4. The transcranial focused ultrasound acoustic pressure field prediction device of claim 1, wherein the transcranial focused ultrasound acoustic pressure field prediction program is provided by artificial intelligence based on a deep neural network (DNN).

5. The transcranial focused ultrasound acoustic pressure field prediction device of claim 4, wherein the transcranial focused ultrasound acoustic pressure field prediction program includes:
an encoder for training a sparse matrix for the shape data of the skull, which is compressed; and
a decoder for receiving the sparse matrix for the skull and the position data of the ultrasonic transducer, which is normalized, to generate an ultrasound acoustic pressure field in a region of interest corresponding to the received sparse matrix and the received position data.

6. The transcranial focused ultrasound acoustic pressure field prediction device of claim 5, wherein the transcranial focused ultrasound acoustic pressure field prediction program is configured based on an autoencoder model based on a convolutional neural network.

7. The transcranial focused ultrasound acoustic pressure field prediction device of claim 5, wherein the transcranial focused ultrasound acoustic pressure field prediction program is configured based on a pix2pix model.

8. The transcranial focused ultrasound acoustic pressure field prediction device of claim 5, wherein the transcranial focused ultrasound acoustic pressure field prediction program is configured based on a variational autoencoder model.

9. The transcranial focused ultrasound acoustic pressure field prediction device of claim 4, wherein the artificial intelligence uses training data including the shape data of the skull, the position data of the ultrasonic transducer, and the ultrasound acoustic pressure field data to perform training so as to predict a shape of an ultrasound acoustic pressure field according to the shape data of the skull and the position data of the ultrasonic transducer by using the shape data of the skull and the position data of the ultrasonic transducer as inputs and using the ultrasound acoustic pressure field data corresponding to the inputs as an output.

10. A transcranial focused ultrasound acoustic pressure field prediction program, wherein the transcranial focused ultrasound acoustic pressure field prediction program is stored in a recording medium that is readable by a transcranial focused ultrasound acoustic pressure field prediction device in order to allow the transcranial focused ultrasound acoustic pressure field prediction device to execute:
inputting shape data of a skull and position data of an ultrasonic transducer, and outputting ultrasound acoustic pressure field data formed in a transcranial region by ultrasound applied by the ultrasonic transducer.

11. The transcranial focused ultrasound acoustic pressure field prediction program of claim 10, wherein the position data of the ultrasonic transducer includes coordinate data representing three-dimensional coordinates at which the ultrasonic transducer is positioned with respect to the skull, and angle data representing an angle at which the ultrasonic transducer is positioned with respect to the skull.

12. A method for implementing acoustic pressure field generation artificial intelligence, which is provided by a transcranial focused ultrasound acoustic pressure field prediction program stored in a recording medium that is readable by a transcranial focused ultrasound acoustic pressure field prediction device, the method comprising:
preparing training data including shape data of a skull, position data of an ultrasonic transducer, and ultrasound acoustic pressure field data; and
allowing artificial intelligence to perform training so as to predict an ultrasound acoustic pressure field formed in a transcranial region by ultrasound applied by the ultrasonic transducer by using the shape data of the skull and the position data of the ultrasonic transducer as inputs and using the ultrasound acoustic pressure field data corresponding to the inputs as an output in the training data.
